(19) United States
(12) Patent Application Publication
(10) Pub. No.: US 2006/0100439 A1
(43) Pub. Date: May 11, 2006

(54) Process for the preparation of modification I of n-(1-methylethylaminocarbonyl)-4-(3-methylphenylamino)-3-pyridinesulfonamide

(75) Inventors: Darko Filic, Zagreb (HR); Miljenko Dumic, Zagreb (HR); Aleksandar Danilovski, Rijeka (HR); Bozena Klepic, Jastrebarsko (HR); Ines Fistric, Zagreb (HR); Marina Marinkovic, Sesvete (HR); Jasna Horvat-Mikulcic, Zagreb (HR)

Correspondence Address: GREENBLUM & BERNSTEIN, P.L.C., 1950 ROLAND CLARKE PLACE, RESTON, VA, 20191, US

(73) Assignee: Pliva Hrvatska d.o.o., Zagreb (HR)

(21) Appl. No.: 10/521,609

(22) PCT Filed: Jul. 7, 2003
(86) PCT No.: PCT/HR03/00036

§ 371 Date: Sep. 30, 2005

(30) Foreign Application Priority Data
Jul. 19, 2002 (HR) P20020603A

(51) Int. Cl. C07D 213/75

(52) U.S. Cl. 546/293

(57) ABSTRACT

The invention relates to a new process for the preparation of modification I of torasemide by precipitation with acids from an alkaline extract of the original reaction mixture of the last phase in the synthesis of torasemide.

The present invention relates to the preparation of modification I of N-(1-methylethylaminocarbonyl)-4-(3-methylphenylamino)-3-pyridinesulfonamide (in the further text of the application designated by its generic name “torasemide”).

Torasemide is a new potent diuretic in the class of so-called “loop” diuretics and is described in DE patent 25 16 025 (Example 71) as 3-isopropylcarbamylsulfonamide-4-(3′-methyl)-phenylamino pyridine. Structurally, it is completely different from diuretics of the same class such as e.g. furosemide, bumetanide and azosemide. Besides diuretic properties it also possesses antihypertensive ones.

As a diuretic of Henle's loop it is interesting as an agent for preventing heart or heart tissue damages caused by metabolic or ionic abnormalities associated with ischemia, in the treatment of thrombosis, angina pectoris, asthma, hypertension, nephroedema, pulmonary edema, primary and secondary aldosteronism, Bartter's syndrome, tumours, glaucoma, decrease of intraocular pressure, acute or chronic bronchitis, in the treatment of cerebral edema caused by trauma, ischemia, concussion of the brain, metastases or epileptic attacks and in the treatment of nasal infections caused by allergens.

Hitherto, some crystal modifications of torasemide have been known: modification I [Acta Cryst. B34(1978), 1304-1310], modification II [Acta Cryst. B34(1978), 2659-2662], modification III (U.S. Pat. No. 6,166,045), modification N (U.S. Pat. No. 6,399,637), modification V (PLIVA; PCTJWO 01/87841), modification V (TEVA; PCT/,WO 01/10441), as well as an amorphous modification of PLIVA (PCTJWO 01/70694), an amorphous modification of TEVA (PCT/WO 01/10441) and Dupont 2 solvate adducts (PCTJWO 01/10441). Crystal modifications I, II and N differ in single cell parameters, which is confirmed by X-ray diffraction on their monocrystals. Modification I with melting point 169° C. [Acta Cryst. B34(1978), 1304-1310] and modification N with melting point 165° C. [U.S. Pat. No. 6,399,637; Croat. Chem. Acta 74(2001) 103-120] crystallize in the monoclinic space group P2₁c (prisms), while modification II with melting point 162° C. crystallizes in the monoclinic space group P2/n (foils) [Acta Cryst. B34 (1978), 2659-2662].

It is known that modification I of torasemide and modification II of torasemide crystallize simultaneously when a torasemide solution in a solvent mixture petroleum ether/ethanol slowly evaporates [Acta Cryst. B34 (1978), 1304-1310]. Such a manner of preparation, however, wherein both modifications crystallize fiom the same solvent mixture and hence must be separated with regard to their macroscopic crystal form, is certainly not suitable for large-scale production.

Further, in the patent application PCT/WO 01/10441 there is described the preparation of modification I of torasemide by recrystallization from methanol of modification II of torasemide or of a mixture of modifications I and II of torasemide or of modification V (TEVA) of torasemide or of Dupont Form 2 solvate adducts of torasemide as well as of their mixtures.

Contrary to the above, in the text of patents U.S. Pat. No. 4,743,693; US reissue 34,580; U.S. Pat. No. 4,822,807; US reissue 34,672; U.S. Pat. No. 5,914,336 and U.S. Pat. No. 6,166,045 it is stated that by recrystallization from solvents always modification II of torasemide is formed. In addition, it is known that by heating torasemide in most solvents its irreversibile decomposition takes place (U.S. Pat. No. 4,743,693; US reissue 34,580; U.S. Pat. No. 4,822,807; US reissue 34,672), whereby the content of accompanying impurities is increased. In view of these statements, recrystallization is not suitable method for the preparation of modification I of torasemide.

The patents U.S. Pat. No. 4,743,693 and US reissue 34,580 protect a process for the preparation of modification I of torasemide, wherein a suspension of modification II of torasemide prepared according to patent DE 25 16 025 in water is stirred under the addition of a catalytic amount of modification I of torasemide (0.1%) at a temperature from room temperature to 90° C. within 3 hours to 14 days. It is stated that no decomposition of torasemide takes place. The patent application PCT/WO 01/10441 discloses the preparation of modification I of torasemide by stiring modification II of torasemide or a mixture of modifications I and II of torasemide in a solvent mixture containing acetonitrile under reflux, or in a mixture of DMSO : acetonitrile at a temperature from 20° C. to 30° C. within 30 to 45 minutes or in a solvent mixture of water: acetonitrile at a temperature from 40° C. to 60° C. over a period of more than 45 minutes. In the same patent application also the preparation of modification I of torasemide by stirring Dupont Form 2 of torasemide in water at pH 5 and at room temperature or by stiring modification II of torasemide in ethanol or dimethylformamide also at room temperature is disclosed.

From the text of patents U.S. Pat. No. 4,743,693; US reissue 34,580; U.S. Pat. No. 4,822,807; US reissue 34,672; U.S. Pat. No. 5,914,336 and U.S. Pat. No. 6,166,045 it is evident that by the preparation and conventional purification of crude torasemide either by precipitation or by recrystallization from solvents the modification II of torasemide is obtained.

In our further investigations in the torasemide field we have surprisingly found a new, hitherto not known process for the preparation of modification I of torasemide.

The new process for the preparation of modification I of torasemide comprises the preparation of modification I of torasemide by directly acidifying an alkaline extract of the original reaction mixture of the last phase in the synthesis of torasemide. The preparation process of the invention enables a more effective, more rapid and economically more acceptable method for the preparation of modification I of torasemide.

According to the process of the invention, for the preparation of the alkaline extract of the original reaction mixture of last phase in the synthesis of torasemide there can be used water solutions of alkaline hydroxides such as solutions of lithium, sodium and potassium hydroxide as well as water solutions of alkaline carbonates such as solutions of sodium and potassium carbonate.

According to the present process, the acidiflying of the alkaline extract of the original reaction mixture of the last phase in the synthesis of torasemide can be performed with inorganic acids such as hydrochloric, sulfuric, phosphoric and nitric acids and with organic acids such as formic, acetic, propionic, oxalic, tartaric, methanesulfonic andp-toluenesulfonic acids and carbon dioxide.

The acidiflying of the alkaline extract of the original reaction mixture of the last phase in the synthesis of torasemide is performed by a continuous addition of the acid under stiring at a stirrer rate from 10 to 300 r/min within 5 minutes to 24 hours until a pH from about 8.5 to about 5.0, most preferably from about 7.5 to about 7.0 is reached, which depends on the acid/base concentration as well as on the batch size, the volume, the construction and the geometry of the reactor. During the addition of the acid it is not necessary to avoid local high concentrations of the acid. The acidifying is performed at temperatures from about 0° C. to about 50° C., most preferably at room temperature.

After reaching the desired pH, the suspension is stirred for another 10 to 240 minutes at a temperature from about 0 to about 50° C., most preferably at room temperature, in order to complete the crystallization. The crystals are separated from the water medium in a usual manner such as filtration, and then dried down to a low moisture content, most preferably below 0.5%.

It has been found that by using of the process of the present invention no decomposition of torasemide occurs, modification I of torasemide contains the remaining solvents within pharmacopeic limits and the impurities that are possibly present in the alkaline extract of the original reaction mixture of the last phase in the synthesis of torasemide pass into the bases, which means that a chemically pure modification I of torasemide is obtained.

Moreover, it has been found that the stable modification I of torasemide prepared according to the process of the present invention is obtained in a “free flow” form, i.e. suitable for the preparation of pharmaceutical forms such as tablets, capsules or injections.

The present invention is illustrated but in no way limited by the following Examples.

### EXAMPLE 1

The original reaction mixture of the last phase in the synthesis of torasemide (starting from 100 g of 4-hydroxy-3-pyridinesulfonic acid) was treated with an about 10% aqueous solution of sodium hydroxide. To the alkaline solution an about 10% aqueous acetic acid solution was continuously added within 30 minutes at room temperature under a stirring rate of about 50 r/min to reach pH 7.0. Then the obtained suspension was stirred for further 30 minutes at the same temperature. The crystals were sucked off and washed with water, whereupon, after drying in a vacuum dryer to a constant weight, 100 g of modification I of torasemide were obtained. Content according to HPLC 99.2%, moisture <0.2%

The IR spectrum and the X-ray powder pattern of the thus obtained sample of modification I of torasemide corresponded to the IR spectrum and the X-ray powder pattern of an authentic sample of modification I of torasemide obtained according to Acta Cryst. B34 (1978), 1304-1310.

### EXAMPLE 2

The original reaction mixture of the last phase in the synthesis of torasemide (starting from 10.0 kg of 4-hydroxy-3-pyridinesulfonic acid) was treated with an about 2.5% aqueous solution of sodium hydroxide. To the solution an about 5% aqueous hydrochloric acid solution was added within 60 minutes at room temperature under a stiring rate of about 200 r/min to reach pH 7.1-7.2. Then the obtained suspension was stirred for further 60 minutes at the same temperature. The crystals were sucked off and washed with water, whereupon, after drying in a vacuum dryer up to a constant weight, 11.0 kg of a stable modification I of torasemide were obtained.

Content according to HPLC 99.1 %, moisture 0.3 %

The IR spectrum and the X-ray powder pattern of the thus obtained sample of modification I of torasemide corresponded to the IR spectrum and the X-ray powder pattern of an authentic sample of modification I of torasemide obtained according to Acta Cryst. B34 (1978), 1304-1310.

1. A process for the preparation of modification I of torasemide, comprising subjecting an alkaline extract of an original reaction mixture of a last phase in the synthesis of torasemide to controlled acidifying with inorganic or organic acid by continuous addition of said acid at room temperature or about room temperature.

2. The process for the preparation of modification I of torasemide according to claim 1, wherein the modification I of torasemide is chemically pure.

3. The process for the preparation of modification I of torasemide according to claim 1, wherein the modification I of torasemide contains less than 0.5% of water.

4. The process for the preparation of modification I of torasemide according to claim 1, wherein the modification I contains remaining solvents within pharmacopeic limits.

5. The process for the preparation of modification I of torasemide according to claim 1, wherein the alkaline extract of the original reaction mixture of the last phase in the synthesis of torasemide is prepared with a water solution of lithium, sodium or potassium hydroxide, and a water solution of sodium or potassium carbonate.

6. The process for the preparation of modification I of torasemide according to claim 1, wherein the acidifying the alkaline extract of the original reaction mixture of the last phase in the synthesis of torasemide comprising acidiying with inorganic acids.

7. The process for the preparation of modification I of torasemide according to claim 1, wherein the acidifying the alkaline extract of the original reaction mixture of the last phase in the synthesis of comprises acidifying with carbon dioxide.

8. The process for the preparation of modification I of torasemide according to claim 1, wherein the acidifying is carried out up to a pH from about 8.5 to about 5.0.

9. The process for the preparation of modification I of torasemide according to claim 8, wherein the acidifying is carried out up to a pH from about 7.5 to about 7.0.

10. The process for the preparation of modification I of torasemide according to claim 1, wherein the acidifying is carried out at a stirrer rate from 10 r/min to 300 r/min.

11. The process for the preparation of modification I of torasemide according to claim 1, wherein the acidifying is carried out within 5 minutes to 24 hours.

12. The process for the preparation of modification I of torasemide according to claim 1, wherein the acidifying is carried out without avoiding high local acid concentrations.

13. The process for the preparation of modification I of torasemide according to claim 1, wherein a suspension obtained after acidifying and reaching the a desired pH is stirred from 10 minutes to 240 minutes.

14. The process for the preparation of modification I of torasemide according to claim 13, wherein the suspension obtained after acidifying and reaching the desired pH is stirred at a temperature from 0° C. to 50° C.

15. The process for the preparation of modification I of torasemide according to claim 14, wherein the suspension obtained after acidifying and reaching the desired pH is stirred at room temperature.
